(19) United States
(12) Patent Application Publication
(10) Pub. No.: US 2004/0224910 A1
(43) Pub. Date: Nov. 11, 2004

(54) Recombinant nematode nicotinic receptor and uses

(76) Inventors: David Sattelle, Oxford (GB); Emmanuel Culetto, Orsay (FR); Howard Baylis, Cambridge (GB)

Correspondence Address: PALMER & DODGE, LLP, KATHLEEN M. WILLIAMS, 111 HUNTINGTON AVENUE, BOSTON, MA, 02199, US

(21) Appl. No.: 10/010,873

(22) Filed: Dec. 7, 2001

Related U.S. Application Data
(63) Continuation-in-part of application No. PCT/GB00/02270, filed on Jun. 9, 2000.

(30) Foreign Application Priority Data
Jun. 9, 1999 (GB) GB99/13248.2

(51) Int. Cl. C12Q 1/68; A01K 67/033; C07H 21/04; C07K 14/705

(52) U.S. Cl. 514/44; 435/6; 435/69.1; 435/320.1; 435/325; 530/350; 536/23.5; 800/8

(57) ABSTRACT

We describe the molecular and functional characterization of the C. elegans unc-63 gene, a levamisole resistance locus on chromosome I, which encodes a nicotinic acetylcholine receptor (nAChR) α subunit. The derived amino acid sequence of UNC-63 most closely resembles that of UNC-38, the product of a separate levamisole resistance locus. Using a gfp::unc-63 fusion construct, expression has been detected in muscles (body wall, vulval) and motorneurons of C. elegans. Nuclear injection into Xenopus laevis oocytes of unc-63 cDNA together with lev-1 and unc-29 results in the expression for the first time of a robust functional C. elegans heteromeric nAChR. The EC₅₀ for ACh of the expressed receptor (20 μM) resembles that of native nematode muscle nAChRs. Nicotine and the anthelmintic drug levamisole are agonists and mecamylamine is an antagonist of this expressed receptor. When unc-38 cDNA is co-injected with cDNAs encoding unc-63, lev-1 and unc-29 much smaller amplitude agonist-activated currents are observed. The first robust functional recombinant heteromeric nAChR composed only of invertebrate subunits has therefore been characterized and shown to contain a pesticide binding site.

The nematode C. elegans has permitted identification and functional analysis of novel genes expressed in the nervous system (Bargmann, 1998). This invertebrate animal provides a highly effective genetic model with which to analyse in vivo molecules involved in chemical synaptic transmission (Jorgensen and Nonet 1995; Rand and Nonet 1997; Sattelle 1998). Neuromuscular cholinergic synapses in C. elegans have been analysed in detail stimulated by the finding that two major postsynaptic components, acetylcholinesterase (ACHE, EC 3.1.1.7) and the nicotinic acetylcholine receptor (nAChR), are targets for widely used anthelmintic drugs. The hydrolytic enzymes AChEs which terminate the actions of ACh are inhibited by carbamates and organophosphates (Massoulie et al 1993). The nAChRs mediate the fast actions of the neurotransmitter ACh. When ACh binds to an nAChR molecule, the receptor molecule becomes transiently permeable to cations (Na⁺, K⁺, Ca²⁺). Anthelmintic drugs such as levamisole, pyrantel and morantel are agonists and open channel blockers at native nematode muscle nAChRs (Martin 1996). The studies to date of cholinergic anthelmintic actions on recombinant ACR-16 (=Ce21) homomeric (probably neuronal) nAChRs (Ballivet et al; Raymond et al 1999) do not mimic the actions observed on native nematode muscle nAChRs.

Five polypeptide subunits surround a central ion channel in each nAChR molecule, each polypeptide having four transmembrane regions (M1-4) and a large N-terminal extracellular domain containing residues that form the ACh binding sites (Karlin, 1993; Unwin 1993; Lena & Changeux 1998). These subunits are classified as either α subunits, possessing two adjacent cysteines in loop C of the ACh binding site or non-α subunits, with no such adjacent cysteine motif.

Radioligand binding studies suggest the possibility of a diversity of nAChRs in C. elegans. For example, using [³H]meta-amino levamisole, a high saturable affinity binding activity has been observed and is regulated in the course of development, the highest binding activity being detected in larval stages (Lewis et al. 1980). Several approaches have been undertaken to characterize further nAChR diversity. For example it is possible to isolate mutants obtained by levamisole drug—resistance selection. The major levamisole resistance loci so far isolated, are as follows: lev-1, lev-8, lev-9, lev-10, lev-11, unc-22, unc-29, unc-38, unc-50, unc-63 and unc-74. The unc-50 gene encodes a product that may possibly be involved in assembly or transcriptional control of receptor units (Lewis et al 1987; Rand and Nonet 1997). The lev-11 and unc-22 genes are involved in muscle contraction, encoding respectively tropomyosin and twitchin (Williams and Waterston 1994, Beniam et al. 1989). Among these 11 resistance loci it has been shown that lev-1 and unc-29 both encode non-α subunit (Fleming et al. 1997), whereas unc-38 encodes an α subunit. Molecular characterization of other loci remains to be done.

A separate genetic screen identified aldicarb resistant mutants. Aldicarb is an ACHE inhibitor. This screen resulted in the isolation of 18 new loci, called ric genes (resistant to inhibitor of cholinesterase) and identified molecular components of both pre-synaptic and post-synaptic terminals (Nguyen et al. 1995, Miller et al. 1996). Of these loci, ric-3 appears to be a good candidate for being a new nAChR subunit.

A genetic approach that screens for reduced pharyngeal pumping has identified other mutants and has resulted in the isolation of two interacting loci (eat-2 and eat-18) which also encode candidate nAChR subunits (Raizen et al. 1995). A further strategy has employed cross—hybridization with either Drosophila nAChR cDNA or previously cloned C. elegans nAChR cDNA. Such techniques have permitted the cloning of three new nAChR subunits acr-2, acr-3 and acr-16 (Squire et al. 1995, Baylis et al. 1997, Ballivet et al. 1996).

Treinen et al (1998) discloses two functionally dependent acetylcholine subunits (des-2 and deg-3) which are encoded in a single C. elegans operon. Their linkage in a single operon allows their coordinated and stoichiometric production in the same cells at the same time. The nAChR subunits DES-2 and DEG-3 are able to form a heteromeric nAChR composed of two different α subunits.

Finally, analysis of the recently (December 1988) completed C. elegans genome sequence shows the presence of 18 new α subunits and 2 new non-α subunits. There is therefore a large family of nAChR subunit genes in C. elegans for which the function remains to be elucidated.

The present invention relates to the cloning, by means of a cross-hybridization approach, of a new α nAChR subunit. We have shown that this new subunit is the product of unc-63, a levamisole—resistant gene. The amino acid sequence of the α nAChR subunit encoded by unc-63 is shown in SEQ ID NO: 1. The present invention further encompasses analogs, variants, mutant forms, derivatives, and fragments of the UNC-63 α nAChR subunit, provided that such analogs, variants, mutant forms, derivatives, and fragments preserve at least one function of the wild-type UNC-63 α nAChR subunit as described herein. UNC-63 is expressed in body wall muscles of C. elegans and in certain motor neurons. This subunit has been co-expressed with UNC-29 and LEV-1 in Xenopus oocytes resulting in the first robust heterologous expression of an invertebrate recombinant heteromeric nAChR. The finding that this expressed nAChR containing UNC-63 mimics several of the properties of native nematode muscle receptors offers new opportunities for in vitro screening of new candidate cholinergic anthelmintic drugs.

Note that the full genomic sequence of C. elegans does not identify the unc-63 gene cDNA sequence as such, and there is no information in the genomic sequence to indicate that a cDNA sequence from C. elegans could permit function recombinant expression of a major anthelmintic drug target (namely the nematode nicotinic acetylcholine receptor) which would effectively mimic the natural receptor.

### FIGURE LEGENDS

FIG. 1. Deduced amino acid sequence of the JTF-38 nAChR α subunit of C. elegans.

amino acids are numbered beginning at the first methionine. Loops contributing to the ACh binding domain are underlined by plain line. The bilayer spanning transmembrane regions TM1-TM4 are underlined by broken lines. The horizontal arrow with a broken line indicates the intrachain di-sulfide bond. The two adjacent cysteines, typical of all nAChR α-like subunits are shown in bold [SEQ ID No. 1].

FIG. 2. Chromosomal localization of the C. elegans jtf-38 gene

Genetic map position of unc-63 on chromosome I. The YACs Y55F5, Y72D6 and Y72E2 span the unc-63 locus.

FIG. 3. Genomic organization of the jtf-38 gene.

The genomic organization of jtf-38 gene is depicted. Boxes indicate exons. SL1 refers to the site of attachment of the trans-splice leader SL1. Three subclones, punc-63.1, punc-63.2 and punc-63.3 of the jtf-38 gene have been tested for their ability to rescue the normal wild type phenotype in unc-63 mutants.

FIG. 4. Dendogram showing UNC-63 and related nAChR subunit family members.

(A) shows C. elegans in relation to other known nicotinic receptor subunits. (B) shows related subunits with C. elegans.

FIG. 5. Amino acid sequence comparison of UNC-63, UNC-38, LEV-1 and UNC-29.

FIG. 6. Functional expression in Xenopus laevis oocytes of UNC-63, UNC-29 and LEV-1 results in a functional nAChR at which ACh is an agonist whereas levamisole and nicotine are partial agonists. The agonist action of levamisole is blocked by 10 μM mecamylamine.

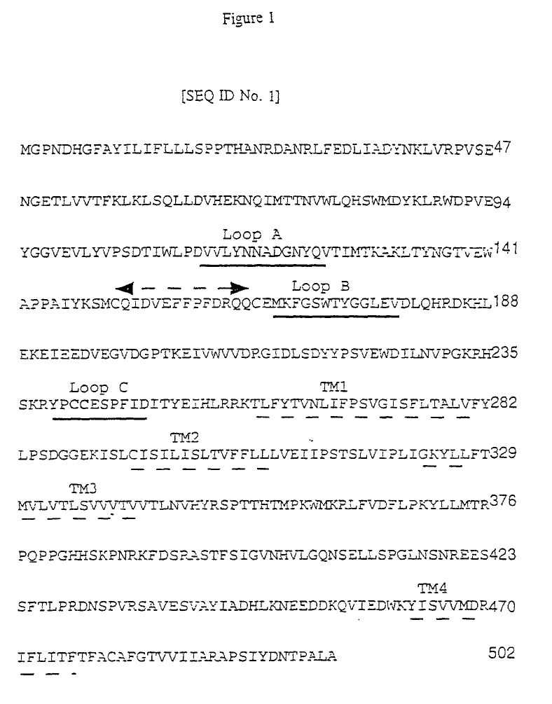

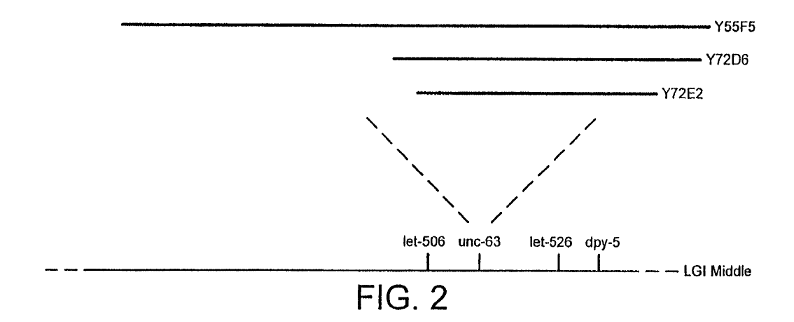

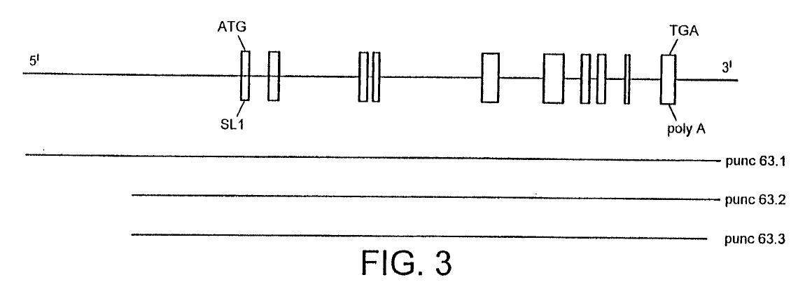

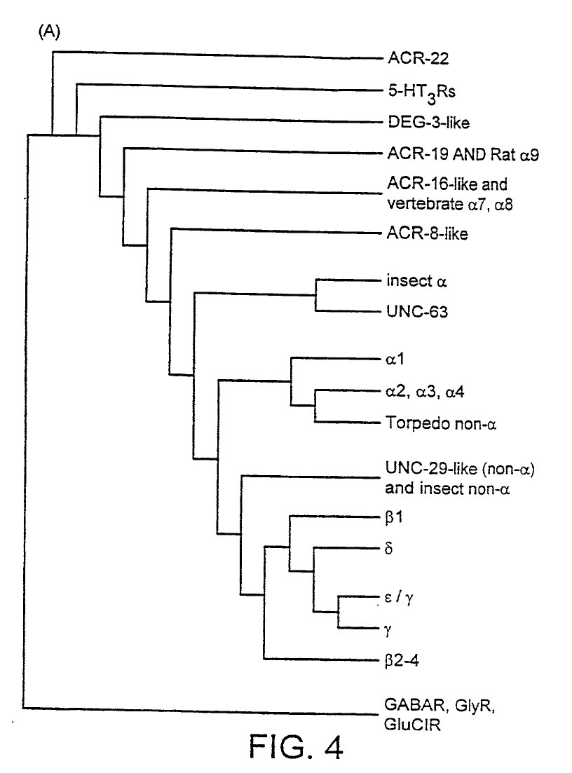

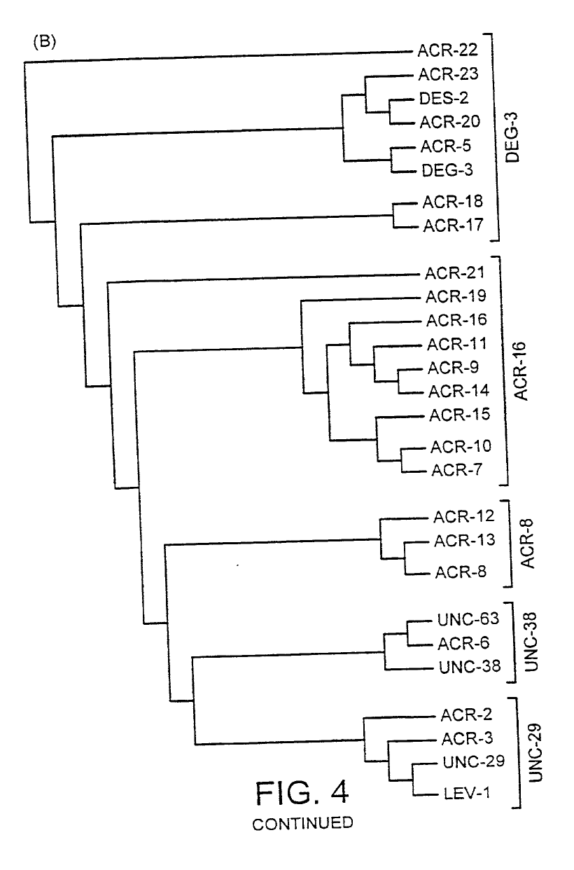

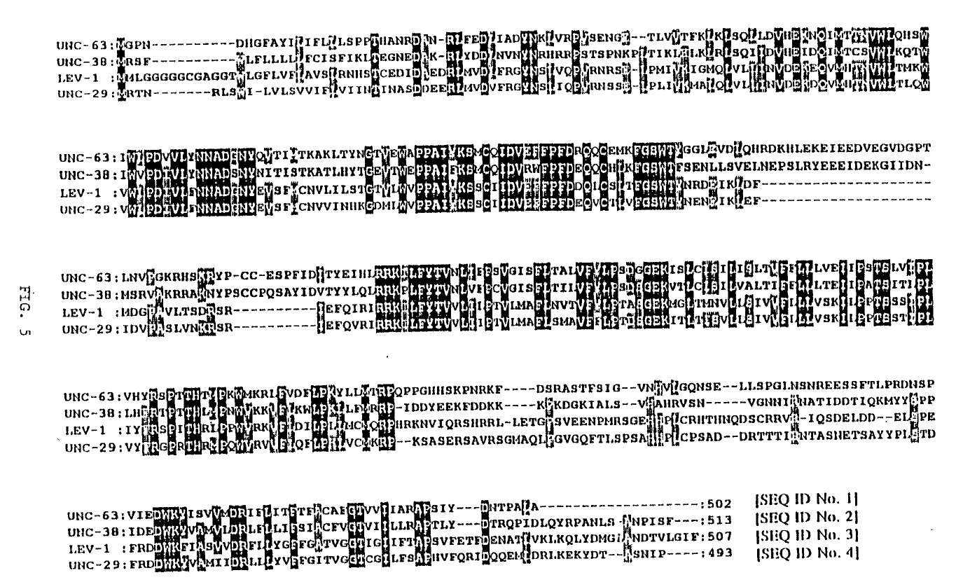

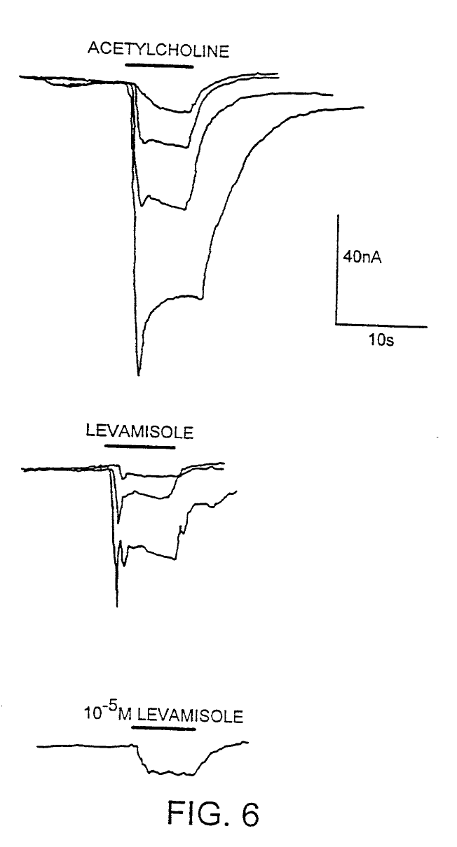

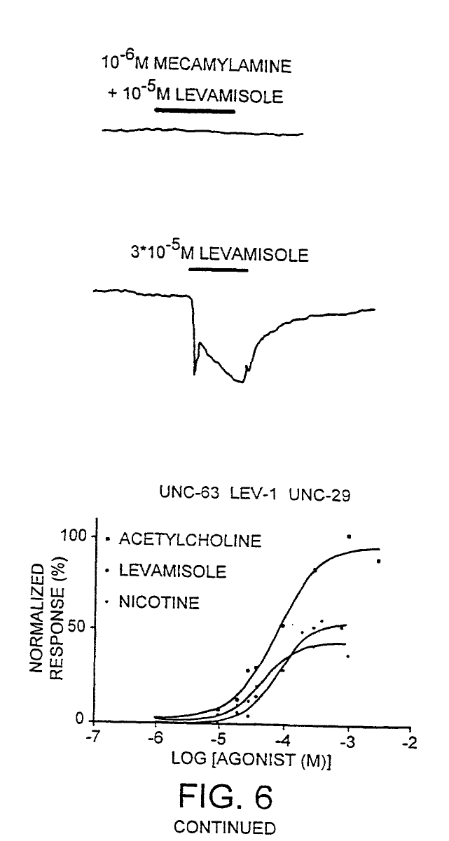

### MATERIALS AND METHODS

C. elegans Strains and General Methods

Worm culture, handling followed the technique described by Sulston and Hodgkin (1988). The wild type C. elegans was the Bristol N2 strain (Brenner, 1974). Strains containing unc-63 alleles zz37, zz26, b404 were obtained from the Caenorhabditis Genetic Center (University of Minessota).

Sequence Analysis

Sequence alignment and analysis were performed with the GCG packaging, CLUSTALW and BLAST.

Molecular Biology

Methods published by Sambrook et al. (1989) were used unless otherwise stated. C. elegans genomic was prepared from wild type as described by Koelle. Plasmid DNA was prepared using Tip 100 from Qiagen. Sequencing was performed according to Sanger et al. with fluorescent dye terminator for automated sequencing.

Cloning of jtf-38 cDNA

A mixed stage cDNA library in kgt10 was screened with a probe for unc-38 (Fleming et al., 1997) at moderate stringency 65° C. in 2×SSC, 0.1% SDS. One of the positive clones JTF38 was subjected to further analysis. Sequencing of a 0.6 kb EcoRI fragment suggested that the clone contained a genomic insert encoding a nAChR subunit.

An antisense oligonucleotide HAB085 was designed which recognized the putative coding sequence from this clone. This oligonucleotide was used in a 5′ RACE reaction using Marathon cDNA (Clontech) derived from total RNA for mixed stage N2 C. elegans. The reaction was carried out using TaqPlus DNA polymerase mix (Stratagene). A band of c. 900 bp was cloned. Sequencing of the ends demonstrated a sequence identical to the JTF38 clone at the 3′ end and a possible nAChR mRNA 5′ end at the other.

An oligonucleotide HAB 112 to the 5′ end was then used to generate a full length cDNA clone by 3′ RACE on Marathon cDNA using Expand polymerase mix (Boehringer Mannheim). A band of 1.6 kb was isolated and cloned into pGEM-T (Promega) to yield the plasmid pHAB385. This insert in this clone was sequenced. The fragment was then excised as a NotI fragment, using sites in the pGEM-T polylinker and in the Marathon cDNA adaptor and ligated into pMT-3. The orientation of clones was established by PCR and the vector insert junction confirmed by DNA sequencing. A clone of the appropriate structure was named pHAB386.

Genetic Localization of jtf-38 Gene

The jtf-38 cDNA was used to probe an ordered grid of yeast artificial chromosome (YAC) clones representing most of the C. elegans genome. The jtf-38 cDNA was labeled by random priming with 50 μCi of ³²P dCTP. Hybridization has been done following the protocol described by Coulson et al. (1995) with 106 cpm/ml of hybridization buffer.

Mutation Detection

Total RNA was isolated using Trizol reagent (Life Technologies) from three different unc-63 allele mutant populations. 2 μg of total RNA was used in each reverse transcription with pdN6 primers, following the manufacturer instructions (Expand reverse transcriptase kit, Boerhinger). Subsequently 5 μl of RT product was used in PCR. We used 4 pairs of primers, deduced from the jtf cDNA sequence, and Taq polymerase from Promega. PCR experiments were run for 40 cycles (denaturation at 95° C. for 1 min, annealing at 55° C. for 1 min and elongation at 72° C. for 1 min). PCR products were cloned into pGEM-T (Promega) and sequenced. Two different clones were sequenced at least for each product. When the mutation was determined the corresponding genomic was sequenced to confirm it. In this case single worm PCR was performed. Briefly 5 mutant worms were picked from the plate and transferred into a lysis buffer containing 1× expand buffer from Boeringher and proteinase K (0.05 mg/ml). Worms were denatured by 1 hour incubation at 65° C. following by 10 min incubation at 95° C. Subsequently 5 μl of lysis worms are used for a PCR using primers deduced from the genomic region showing the mutation.

Germline Transformation

Germ line transformation was performed according to the method of Mello and Fire (1995).

Mutant Rescue Experiment:

For rescue experiments 3 different constructs, punc-63.1, punc-63.2 and punc-63.3 were generated by means of PCR using the Expand Long Template System (Boeringher) on genomic DNA isolated from N2 worms as described by Koelle (1988). PCR products were cloned into the pGEM-T plasmid. Clones were subsequently checked for the presence of known restriction sites and by sequencing both 5′ and 3′ extremities. The punc-63.1 construct contains a 12.5 kb insert comprising 4.5 kb of 5′ region, the all genomic coding region and 1 kb of 3′ untranslated sequence punc-63.2 contains a 10 kb insert and differs from punc-63.1 in that it has only 2.5 kb of genomic sequence upstream of the unc-63 ATG site. The punc-63.3 construct differs from punc-63.2 only by 0.7 kb of 3′ untranslated sequence. Germ line transformation was performed by co-injecting the test DNA at a concentration of 100-120 ng ml⁻¹ and the plasmid pPD93 65 which contains the GFP gene under the control of the promoter for unc-54, the myosin heavy chain gene expressed in all muscle cells. Transgenic animals are therefore selected by GFP fluorescence in body wall muscle cells and grown on individual plates enabling studies on the phenotype of rescued worms (levamisole sensitivity, normal locomotion and egg laying).

GFP Localisation:

A 10.6 kb fragment was amplified from genomic DNA by means of PCR, using the Expand Long Template system (Boeringher), with primers designed to contain SphI (sense primer) and XmaI (forward primer) restriction sites at one end. The fragment includes 4.5 kb of the putative 5′ regulatory region and 6 kb of genomic coding sequence including exon I through part of exon 7 (encoding the TM3-TM4 extracellular loop). This fragment has been cloned in frame into the GFP expressing vector pPD95.70 using the engineered restriction site at both ends of primers. This construction has been designated UNC-63::GFP1.

The fusion construct UNC-63::GFP1 at a concentration of 80 mg ml⁻¹ was co-injected with plasmid pRF4 (10 ng μl⁻¹) into wild type animals. Transgenic animals were selected by their roller phenotype and viewed by fluorescence microscopy (Zeiss Axiovert 35 filtersX).

Functional Expression in Xenopus laevis oocytes

Ovaries were surgically removed from anaesthetised mature female Xenopus laevis. The follicle layers were manually removed from healthy stage V and VI oocytes following a 15 min incubation with collagenase (type IA, 2 mg ml⁻¹) in a calcium-free version of standard oocyte saline. The composition of SOS was as follows (mM): NaCl 100, KCl 2, CaCl₂ 1.8, MgCl₂ 1 and HEPES 5; pH 7.6. In calcium-free saline, the CaCl₂ was replaced by 1.8 mM BaCl₂. The UNC-63-pMT₃, UNC-29-pMT3, LEV-1-pMT₃ and UNC-38-pMT₃ expression constructs (Swick et al., 1992) were isolated from E. coli JM109 using endo-toxin free maxi-prep kits (Qiagen). The nucleus of each oocyte was injected with 20 nl of DNA (0.1 μg μl⁻¹). The injected oocytes were transferred to incubation medium composed of SOS supplemented with penicillin (100 units ml⁻¹), streptomycin (100 μg ml⁻¹), gentamycin (50 μg ml⁻¹) and 2.5 mM sodium pyruvate and placed at 4° C. for 30 min immediately after injection to enable recovery. Oocytes were maintained at 16° C. for 2-4 days prior to electrophysiological studies.

Electrophysiology

Oocytes were restrained with entomological pins in a Perspex chamber (80 μl volume) with a Sylgard base and perfused continuously (5 ml min⁻¹) with SOS using a gravity-fed system. Membrane currents were measured by the two-electrode voltage-clamp method, using 3M KCl filled electrodes (resistance=0.5-5 MΩ) and either a Geneclamp amplifier (Axon Instruments). The oocyte membrane potential was clamped at −100 mV. Signals were digitized by a TL-1 interface (Axon Instruments, U.S.A).

Chemicals

Acetylcholine chloride, nicotine, levamisole and mecamylamine were obtained from Sigma. Unless otherwise indicated all other chemicals were obtained from Sigma (UK).

Results

Cloning a Novel C. elegans nAChR Subunit

A C. elegans cDNA phage (λ7) library was screened at low stringency using the unc-38 and unc-29 cDNAs as probes. Several positive clones were obtained. One positive clone, jtf-38, hybridized specifically at low stringency with unc-38 and was investigated further. This positive clone was a partial cDNA but showed 50% identity at the amino acid level with UNC-38. We then utilised 5′RACE to identify the 5′ end of the mRNA and showed that the transcript was transpliced at its 5′ end to the RNA splice leader SL1 (Krause and Hirsh, 1987). The SL1 sequence was found upstream the putative methionine ATG. We then used 3′RACE to amplify a full length cDNA clone. This cDNA was designed jtf-38.

The jtf-38 Clone Encodes a Nicotinic Acetylcholine Receptor Subunit

Sequence analysis of clone jtf-38 revealed an open reading frame of 502 amino acids. The presumptive deduced protein has a calculated molecular weight of 65 kDa. The sequence shows all the characteristics (cf Galzi and Changeux 1995, Hucho et al 1996) of a new nAChR α subunit. We found conserved stretches of amino acids involved in the ACh binding site including loop A, loop B and loop C motifs as well as the ‘cys loop’ defined by the di-sulfide bridge between cysteines 151 and 165. The two adjacent cysteines, typical of α subunits, are located at amino acid positions 241 and 242. In contrast to UNC-38 (Fleming et al., 1997) and several other C. elegans nicotinic receptor (Mongan et al., 1999), the JTF-38 subunit had the typical Y-X-C-C motif in the putative loop C of the ACh binding site.

There are 4 putative transmembrane domains, TM1 (260-283), TM2 (291-308), TM3 (324-344) and TM4 (464-476). The GCG MOTIFS programidentified 3 putative phosphorylation sites all included into the intracellular loop TM3-TM4. It has been shown that the Torpedo receptor can be phosphorylated by at least three different protein kinases: cAMP—dependant kinase (PKA), protein kinase C (PKC) and a tyrosine kinase (TK). The putative sites on unc-63 are of the PKC (2) and TK (1) type.

Physical and Genetic Locations of the C. elegans Gene Identified by the jtf-38 Clone

We mapped the physical location of this new nAChR gene to YACs Y55F5 and Y72D6, in a cosmid gap, by hybridizing the cloned cDNA to the YAC grid. Thus the jtf-38 gene maps to the center of chromosome I. In this genomic region defined by these two overlapping YACs, lies the levamisole resistant loci unc-63. On the other hand previous work has shown that a small YAC Y72E2, which overlaps partially Y55F5 and completely Y72D6, has been injected in the mutant unc-63 and rescued the levamisole resistance conferring unc-63 mutant (T. Barnes, personal communication). We therefore tested whether JTF-38 was unc-63. We thus determined the entire coding sequence of the corresponding JTF-38 cDNA in three different unc-63 mutant alleles.

Several Classes of unc-63 Mutations

We generated jtf-38 cDNA by RT-PCR from three different mutant alleles of unc-63. The x37 mutant allele worms are inactive, slow and extremely resistant to levamisole. For this allele we found a single base transition G:C to A:T at the flanking region of intron 4. The intron 4 is not correctly cis-spliced, changing the open reading frame and introducing a stop codon in-frame.

The b404 mutant allele has a slight levamisole resistance and a slight uncoordinated movement. We found a deletion (138 nt) in the coding sequence of the M3-M4 intracellular loop. This deletion kept in phase the two remaining fragments. This deletion removes conserved amino acids and at least the three putative phosphorylation sites. Moreover it has been recently showed, in vivo, that this large intracellular loop is also involved in the localization of nAChR to the active site at the synapse (Williams et al., 1998).

The x26 mutant allele has normal movement and slight levamisole resistance. We found a G:C to A:T transition changing the cysteine 151 to a Tyrosine. This cysteine part of the loop B involves in the ACh binding site. This same mutation has been shown recently to be involved in a human congenital myasthenia syndrome (Milone et al 1998). The mutant receptor subunit fails to incorporate into the cell surface and is therefore a null mutation.

Thus the findings for the unc-63 mutant alleles provide evidence that the nAChR ∀ subunit cDNA we cloned and unc-63 are the same gene. In the course of this study the complete genomic sequence of YAC 72E2 covering the unc-63 locus was determined by the Sanger Center. We therefore compared the unc-63 genomic and cDNA sequences. The unc-63 gene is composed of 10 exons spanning 7.5 kb. To examine the expression of unc-63 we performed a Northern blot hybridization and detected a single transcript.

unc-63 is Expressed in Both Muscle Cells and Neurons

To address the question of the localization of unc-63 we monitored its expression by fusing to GFP (Green Fluorescent Protein) (Chalfie et al., 1994) a genomic region comprising 4.5 kb of 5′ upstream, promoter—containing sequence and the genomic unc-63 coding region encompassing the first 7 exons. In transgenic animals expressing the UNC-63::GFP construct, fluorescent signals were observed in all body wall muscle cells and in vulval muscle cells. We also found expression in many cells of the nervous system, including motor neurons. These findings are consistent with the unc-63 mutant defects as mutant worms have defects in locomotion and exhibit abnormal egg laying rate. This expression pattern suggests that unc-63 functions in both muscle and nerve cells of C. elegans.

The C. briggsae Genome Contains a Very Close Relative of the C. elegans unc-63 Gene.

The divergence between the two closely related nematode Caenorhabditis elegans and Caenorhabditis briggsae is as large as between mammals and reptiles (Fitch et al., 1995). Many proteins have both a very high level of conservation in sequence (Grauso et al. 1996) and in function between the two species (Kennedy et al., 1993; Krause et al., 1994). Using PCR and primers deduced from the C. elegans unc-63 sequence we amplified a nearly full length cDNA. The high homology (95%) between the 2 sequences strongly suggests that we have cloned the C. briggsae unc-63 homologue.

Functional Expression of unc-63 in Xenopus oocytes

When cDNAs encoding unc-63, lev-1 and unc-29 were injected separately into Xenopus laevis oocytes no evidence of ACh—induced currents was obtained. Pairwise injections of all combinations were similarly unsuccessful in generating ACh—induced currents. However, when all 3 cDNAs were co—injected robust ACh—induced currents (inwardly directed) were detected at a holding potential (Eh) of −100 mV. The ACh dose—response curve resulted in EC₅₀ value of 20 μM. Whereas ACh was a full agonist, levamisole and nicotine showed partial agonist activity on the expressed heterotrimeric UNC-63, UNC-29, LEV-1 receptor. Mecamylamine (10 μM) was an effective antagonist of the ACh—induced currents 5recorde from the expressed UNC-63, UNC-29, LEV-1 heterotrimeric receptor. Thus this robust recombinant heteromeric nAChR resembles in respect of ACh, nicotine and levamisole and mecamylamine actions the native muscle nAChR of Ascaris suum muscle (Colquhoun et al., 1991; 1993).

Discussion

UNC-63 is a new C. elegans nicotinic acetylcholine receptor (nAChR) α subunit. Its amino acid sequence shows the vicinal cysteine motif by which such subunits are defined. There are 4 putative transmembrane subunits, a long N-terminal region containing sites that show strong conservation with the loops (A-F) which appear to make up ACh binding site (Lena and Changeux 1998). In the case of UNC-63 the vicinal cysteines are part of a Y-X-C-C motif in loop C. Based on its amino acid sequence homology UNC-63 is designated a member of the UNC-38-like nAChR subunit family, all members of which identified to date are ∀ subunits. Using a gfp fusion construct we have shown that UNC-63 is expressed in all body wall muscles, in vulval muscles and in certain motor neurons of C. elegans. UNC-63 and UNC-29 are both strongly expressed in body wall muscle of C. elegans (as well as in certain neurons) but nothing is known to date of the expression of LEV-1. This pattern of spatial distribution with a nAChR subunit not being confined to a particular cell or tissue type has now been found for several C. elegans nAChR subunits (see Table 1), a situation that contrasts strikingly with the situation that so far obtains in vertebrates where separate gene families are expressed in nerve and muscle.

UNC-63 when co-expressed in Xenopus oocytes with UNC-29 and LEV-1 results in the most robust functional expression observed to date for a C. elegans recombinant heteromeric nAChR and indeed for any recombinant heteromeric nAChR containing only invertebrate subunits. For example, earlier work on C. elegans recombinant nAChRs using the UNC-38, UNC-29 and LEV-1 combination did result in functional heteromeric receptor but the current amplitudes were much lower than those reported here. Interestingly when we co-expressed UNC-38 with the the other 3 subunits used throughout the present study, this reduced the amplitude of the currents recorded. It is not clear why UNC-38 has this effect. It does have an unusual Y-X-X-C-C motif in loop C of the ACh binding site which may conceivably impair normal ACh—receptor interactions but other factors also remain to be investigated. Certain neuronal nAChR receptors of vertebrates are known to contain 2 distinct ∀ subunits notably ∀3 and ∀5. The ∀5 subunit also has an atypical (A-X-C-C) loop C motif. It may be that UNC-63 and UNC-38 are not normally expressed in the same nAChR molecule. The possibility that another non-∀ subunit is required for UNC-38 to exert its full functional role cannot be discounted. A possible functional role may exist for a ‘silencing subunit’ eg early in development or at the dauer stage. Changeux and colleagues have suggested a labelling role for nAChRs in synapse formation early in development. Alternatively, this effect may simply be the result of mis-assembly of subunits that don't normally belong together.

Thus of the 11 genes linked to levamisole resistance 4 are now known to be nAChR subunits and one other remains a possible candidate.

The gene unc-63 encoding the new ∀ subunit described here is located on chromosome I of C. elegans. The 2 other known members of the UNC-38-like group of ∀ subunits are located on the same chromosome. None are in sufficiently close proximity to be part of a common transcription unit as is the case for other ∀ subunit genes such as (deg-3, des-2).

Mutants of unc-63 have proved to be instructive. The mis-sense mutation in allele x37 which results in the insertion of a stop codon gives rise to a phenotype showing very strong resistance to levamisole and slow movement. In the x26 mutant allele, which appears to have normal movement and only slight levamisole resistance, the cysteine at position 151 is replaced by a tyrosine. The effect of this change in the N-terminal region (part of loop B) is to open up the cys loop, an effect similar to that produced by one of the ∀ subunit mutations resulting in a congenital myasthenic syndrome.

### CONCLUSION

This new ∀ subunit has permitted the first robust functional expression of a nematode nAChR on which levamisole has similar actions to those observed on native nematode muscle nAChRs. This transient expression system and in future a stable cell line containing such recombinant receptors offers the prospect for the first time of rapid high—throughput screening for a new generation of cholinergic anthelmintics and endectocides.

TABLE 1

| Subunit | Location of expression | Method | References |
|---|---|---|---|
| deg-3, | PVC, PVD, FLP (touch | LAC-Z | Treinin and |
| des-2 | neurons) IL2 neuron head | reporter gene | Chalfie, 1995 |
| | muscle cell | | Treinin et al., |
| | | 1999 | |
| unc-29 | Body wall muscle cells | GFP | Fleming et |
| | head neurons | reporter gene | al., |
| | | | 1997 |
| acr-5 | B-type motor neurons | GFP | J. Ahringer, |
| | | reporter gene | pers. |
| | | | comm.. |
| acr-2 | Motor neurons (multiple | GFP | H. R. |
| | classes) | reporter gene | Horwitz, |
| | | | pers. comm. |
| unc-38 | Muscle cells and neurons | Genetic | H. R. |
| | | interactions of | Horwitz, |
| | | unc-38 with both | pers. comm. |
| | | unc-29 and acr-2 | |
| | | mutants | |
| unc-63 | Body wall muscle and | GFP | E. Culetto, |
| | Motor neurons (multiple | reporter gene | pers. comm. |
| | classes) | | |

### References

Ballivet M, Alliod C, Bertrand S, Bertrand D (1996) Nicotinic acetylcholine receptors in the nematode Caenorhabditis elegans. J. Mol. Biol. 258:261-269.

Bargmann, C.I. (1998) Neurobiology of the Caenorhabditis elegans genome. Science 282: 2028-2033.

Baylis, H. A., Matsuda, K., Squire, M. D., Fleming, J. T., Harvey, R., Darlison, M. G., Barnard, E. A., and Sattelle, D. B. (1997) ACR-3, a Caenorhabditis elegans nicotinic acetylcholine receptor subunit: molecular cloning and functional expression. Receptors and Channels 5: 149-158

Benian, G. M., Kiff, J. E., Neckelmann, N., Moerman, D. G. and Waterston, R. H. (1989) Sequence of an unusually large protein implicated in regulation of myosin activity in C. elegans. Nature 342: 45-50.

Chalfie, M., Tu, Y., Euskirchen, G., Ward, W. W. and Prasher, D. C. (1994) Green fluorescent protein as a marker for gene expression. Science 263: 802-805.

Colquhoun, L., Holden-Dye, L. and Walker, R. J. (1991) The pharmacology of cholinoreceptors on the somatic muscle cells of the parasitic nematode Ascaris suum. J. Exp. Biol. 158, 509-530.

Colquhoun, L., Holden-Dye, L. and Walker, R. J. (1993) The action of nicotinic receptor specific toxins on the somatic muscle cells of the parasitic nematode Ascaris suum. Mol. Neuropharmacol. 3, 11-16.

Coulson, A. R., Huynh, C., Kosono, Y. and Shownkeen, R. (1995) The physical map of The Caenorhabditis elegans genome. In Caenorhabditis elegans. Modern Biological Analysis of an Organism, pp 534-549, Academic Press.

Fitch, D. H. A, Bugaj-Gaweda, B. and Emmons, S. W. (1995) 18S ribosomal RNA gene phylogeny for some Rhabditidae related to Caenorhabditis elegans. Mol. Biol. Evol. 12: 346-358.

Fleming, J. T., Squire, M. D., Barnes, T. M., Tornoe, C., Matsuda, K., Ahnn, J., Fire, A., Sulston, J. E., Barnard, E. A., Sattelle, D. B. and Lewis, J. A. (1997) J. Neurosci. 17: 5843-5857.

Galzi, J. L., and Changeux, J. P., (1994) Neurotransmitter-gated ion channels as unconventional allosteric proteins. Curr. Opin. Struc. Biol 4: 554-565.

Grauso, M., Culetto, E., Berge, J. B., Toutant, J. P. and Arpagaus, M. (1996) Sequence comparison of ace-1 the gene encoding acetylcholinesterase of class A, in the two nematodes Caenorhabditis elegans and Caenorhabditis briggsae. DNA sequence 6: 217-227.

Harrow, I. D. and Gration, K. A. F. (1985) Mode of action of the anthelmintics morantel, pyrantel, and levamisole on muscle cell membrane of the nematode Ascaris suum. Pestic. Sci. 16: 662-672.

Hucho, F., Tsetlin, V. I. and Machold, J. (1996) The emerging three dimensional structure of a receptor: The nicotinic acetylcholine receptor. Eur. J. Biochem. 239: 539-557.

Jorgensen, E. M. and Nonet, M. L. (1995) Neuromuscular junctions in the nematode Caenorhabditis elegans. Seminars in Devel. Biol. 6: 207-220.

Karlin A (1993) Structure of nicotinic acetylcholine receptors. Curr. Opin. Neurobiol. 3:299-309.

Kennedy, B. P., Aamodt, E. J., Allen, F. L, Chung, M. A., Heschl, M. F. P. and McGhee, J. D. (1993) The gut esterase gene (ges-1) from the nematodes Caenorhabditis elegans and Caenorhabditis briggsae. J. Mol. Biol. 229: 890-908

Krause, M., Harrison, S. Q., Xu, L., Chen, L. and Fire, A. (1994) Elements regulating cell and stage specific expression of the C. elegans MyoD family homolog hlh-1. Devel. Biol. 166: 133-148.

Krause, M. and Hirsh, D. (1987) A trans-spliced leader sequence on actin mRNA in C. elegans. Cell 49: 753-761.

Lena, C. and Changeux, J. P. (1998). Allosteric nicotinic receptors, human pathologies. J. Physiol. (Paris), 92: 63-74.

Lewis, J. A., Wu, C. H., Levine, J. H., and Berg, H., (1980) Levamisole-resistant mutants of the nematode Caenorhabditis elegans appear to lack pharmacological acetylcholine receptors. J. Neurosci. 5: 967-928.

Lewis, J. A., Elmer, J. S., Skimming, J., McLafferty, S., Fleming, J. and McGee, T. (1987) Cholinergic receptor mutants of the nematode Caenorhabditis elegans. J. Neurosci. 7: 3059-3071.

Martin, R. J., Valkanov, M. A., Dale, M. E., Robertson, A. P. and Murray, I. (1996) Electrophysiology of Ascaris muscle and anti-nematodal drug action. Parasitol. 113: S137-S156.

Massoulié, J., Pezzementi, L., Bon, S., Krjci, E. and Vallette, F. M. (1993) Molecular and cellular biology of cholinesterases. Prog. Neurobiol. 41: 31-91.

Mongan, N. P., Baylis, H. A., Adcock, C., Smith, G. R., Sansom, M. S. P. and Sattelle, D. B. (1998) An extensive and diverse nicotinic acetylcholine receptor V subunit gene family in Caenorhabditis elegans. Receptors and Channels 6: 213-228.

Milone M, Wang H-L, Ohno K, Prince R, Fukudome T, Shen X-M, Brengman J M, Griggs R C, Sine S M, Engel A G. (1998) Mode switching kinetics produced by a naturally occuring mutation in the cytoplasmic loop of the human acetylcholine receptor ε subunit. Neuron 20:575-588.

Miller, K. G., Alfonso, A., Nguyhen, M., Crowell, J. A., Johnson, C. D. and Rand, J. B. (1996) A genetic selection for Caenorhabditis elegans synaptic transmission mutants. Proc. Natl. Acad. Sci. 93: 12593-12598.

Nguyen, M., Alfonso, A., Johnson, C. D. and Rand, J. B. (1995) Caenorhabditis elegans mutants resistant to inhibitors of acetylcholinesterase. Genetics 140: 527-535.

Fleming, J. T., Squire, M. D., Barnes, T. M., Tornoe, C. T., Matsuda, K., Sulston, J. E., Barnard, E. A., Sattelle, D. B., and Lewis, J. T. (1997) Caenorhabditis elegans levamisole resistance genes lev-1, unc-29 and unc-38 encode functional nicotinic acetylcholine receptor subunits. J. Neurosci. 17: 5843-5857.

Rand J B and Nonet M L (1997) “Synaptic transmission” in C elegans II (eds Riddle D L Blumenthal T Meyer B J and Priess J R) pp. 611-643 Cold Spring Harbor Laboratory Press.

Raizen, D. M., Lee, R. Y. N. and Avery, L. (1995) Interacting genes required for pharyngeal excitation by motor neuron MC in Caenorhabditis elegans. Genetics 141: 1365-1382.

Raymond, V., Mongan, N. P. and Sattelle, D. B. (1999) Actions of cholinergic anthelmintics and ivermectin on recombinant homomeric nicotinic acetylcholine receptors, chicken 7 and Caenorhabditis elegans ACR-16. Brit. J. Pharm. (submitted).

Sattelle, D. B. (1998). Genetic, genomic and functional studies on the nicotinic acetylcholine receptor gene family of Caenorhabditis elegans. J. Physiol. 18S: 513P.

Unwin, N. (1993) Nicotinic acetylcholine receptor at 9A resolution. J. Mol. Biol. 229: 1101-1124.

Thompson, J. D. Gibson, T. J. Plewniak, F., Jeamnougin, F. and Higgins, D. G. (1997) The CLUSTAL_X windows interface: flexible strategies for multiple sequence alignment aided bu quality analysis tools. Nucleic Acids Res. 25: 4876-4882.

Treinin, M. et al (1988) Two functionally dependent acetylcholine subunits are encoded in a single C. elegans operon. P.N.A.S. 95. 15492-15495.

Williams, B. D. and Waterston, R. H. (1994) Genes critical for muscle development function in Caenorhabditis elegans identified through lethal mutations. J. Cell Biol. 124: 475-490.

1. A recombinant nematode nicotinic receptor comprising the amino acid sequence set forth in SEQ ID No.:1.

2. An isolated DNA which encodes a C. elegans unc-63 α nAChR subunit, which subunit is a functional nematode nicotinic receptor, comprising the amino acid sequence of SEQ ID No. 1.

3. A DNA segment according to claim 2 which is a cDNA comprising the amino acid sequence of SEQ ID No.:1.

4. A vector containing the DNA of claim 2.

5. A host transformed by the vector of claim 4.

6. A host containing a transgene encoding a recombinant receptor according to claim 1.

7. A host according to claim 6 which is a cell line.

8. A method of producing a recombinant nematode nicotinic receptor having the amino acid sequence set forth in SEQ ID No.:1 comprising culturing a host cell according to claim 6 under conditions which permit the expression of said receptor.

9. A method according to claim 8 in which the C. elegans unc-63 gene which encodes α nAChR subunit is coexpressed with one or more nAChr subunits.

10. A method of screening for antihelmintic compounds which includes the steps of:
i) exposing a recombinant receptor according to claim 1 to a compound to be screened for antihehnintic activity;
ii) selecting a compound which interacts with said receptor; and
iii) characterising said selected compound as an antihelmintic compound.

11. A method of controlling parasitic nematode growth in a host, which comprises the administration of:
i) exposing a recombinant receptor according to claim 1 to a compound to be screened for antihelmintic activity;
ii) selecting a compound which interacts with said receptor; and
iii) characterising said selected compound as an antihelmintic compound.

12. A recombinant nematode nicotinic receptor according to claim 1 which mimics the response of the natural receptor to the antihelmintic drug Levamisole.
